(19) United States
(12) Patent Application Publication
(10) Pub. No.: US 2019/0076042 A1
(43) Pub. Date: Mar. 14, 2019

(54) MYOELECTRICITY MEASUREMENT DEVICE

(71) Applicants: KABUSHIKI KAISHA TOSHIBA, Tokyo (JP); TOSHIBA ELECTRONIC DEVICES & STORAGE CORPORATION, Tokyo (JP)

(72) Inventors: Kazuyuki TAKAYAMA, Kisarazu Chiba (JP); Manabu SAKAI, Yokohama Kanagawa (JP)

(21) Appl. No.: 15/909,223

(22) Filed: Mar. 1, 2018

(30) Foreign Application Priority Data
Sep. 13, 2017 (JP) 2017-175387

(51) Int. Cl. A61B 5/0408; A61B 5/0428; A61B 5/11; A61B 5/00

(52) U.S. Cl. CPC A61B 5/0408; A61B 5/6802; A61B 5/1107; A61B 5/0428

(57) ABSTRACT

A myoelectricity measurement device includes a plurality of myoelectricity sensor electrodes, a measurement unit connectable to the plurality of myoelectricity sensor electrodes, and a control unit configured to select a first combination of at least three myoelectricity sensor electrodes to be connected to the measurement unit, designate a first electrode in the first combination as a reference electrode, a second electrode in the first combination as a first input electrode, and third electrode in the first combination as a second input electrode, acquire a first voltage difference between the first and second input electrodes in reference to a voltage of the reference electrode, the voltage difference, and select a second combination of at least three myoelectricity sensor electrodes to be connected to the measurement unit, at least one myoelectricity sensor electrode in the second combination being different from the myoelectricity sensor electrodes in the first combination.

### CROSS-REFERENCE TO RELATED APPLICATION

This application is based upon and claims the benefit of priority from Japanese Patent Application No. 2017-175387, filed Sep. 13, 2017, the entire contents of which are incorporated herein by reference.

### FIELD

Embodiments described herein relate generally to a myoelectricity measurement device.

### BACKGROUND

In existing myoelectricity measurement devices, multiple sensor electrodes in contact with the skin of a user are used to detect electrical signals and these electrical signals are analyzed to evaluate the user's muscle movement. However, since locations of muscles are different for each user, the intended data may not be acquired even if an increased number of myoelectricity sensor electrodes are adopted. Generally, elastic elements, such as wristbands, belts, or wraps, are used to keep the myoelectricity sensor electrodes in a fixed position. However, such elastic elements expand or contract according to a user's movement, and the positions of the sensor electrodes may be shifted as a result.

Therefore, there is a need for myoelectricity measurement devices that can acquire intended data with high precision even though the myoelectricity sensor electrodes are shifted from intended positions.

### DESCRIPTION OF THE DRAWINGS

FIG. 1 is a diagram of a myoelectricity measurement device according to a first embodiment.

FIG. 2 depicts measurement of myoelectricity and a relationship between voltages at sensor electrodes.

FIG. 3 is a flowchart of a myoelectricity measurement process.

FIG. 4 is a schematic diagram of a myoelectricity measurement device.

FIG. 5 depicts an enlarged view of sensor electrodes and example combinations of sensor electrodes.

FIG. 6 depicts an example of a circuit of a selection unit.

FIG. 7 depicts another example arrangement of sensor electrodes in a zigzag form and example combinations of measurement sensor electrodes.

FIG. 8 depicts another example arrangement of sensor electrodes.

FIG. 9 depicts still another example arrangement of sensor electrodes.

FIG. 10 depicts a myoelectricity measurement device according to a second embodiment.

### DETAILED DESCRIPTION

In general, according to one embodiment, a myoelectricity measurement device includes a plurality of myoelectricity sensor electrodes, a measurement unit connectable to the plurality of myoelectricity sensor electrodes, and a control unit configured to select a first combination of at least three myoelectricity sensor electrodes from the plurality of myoelectricity sensor electrodes to be connected to the measurement unit, designate a first electrode in the first combination as a reference electrode, a second electrode in the first combination as a first input electrode, and third electrode in the first combination as a second input electrode, acquire a first voltage difference between the first and second input electrodes in reference to a voltage of the reference electrode, the voltage difference being measured by the measurement unit, and select a second combination of at least three myoelectricity sensor electrodes from the plurality of myoelectricity sensor electrodes to be connected to the measurement unit, at least one of the three myoelectricity sensor electrodes in the second combination being different from the at least three myoelectricity sensor electrodes in the first combination.

Hereinafter, a myoelectricity measurement device according to example embodiments will be described in detail with reference to the drawings. It should be noted that the particular embodiments explained below are some possible examples of a myoelectricity measurement device according to the present disclosure and do not limit the possible configuration, specifications, or the like of myoelectricity measurement devices according to the present disclosure.

### First Embodiment

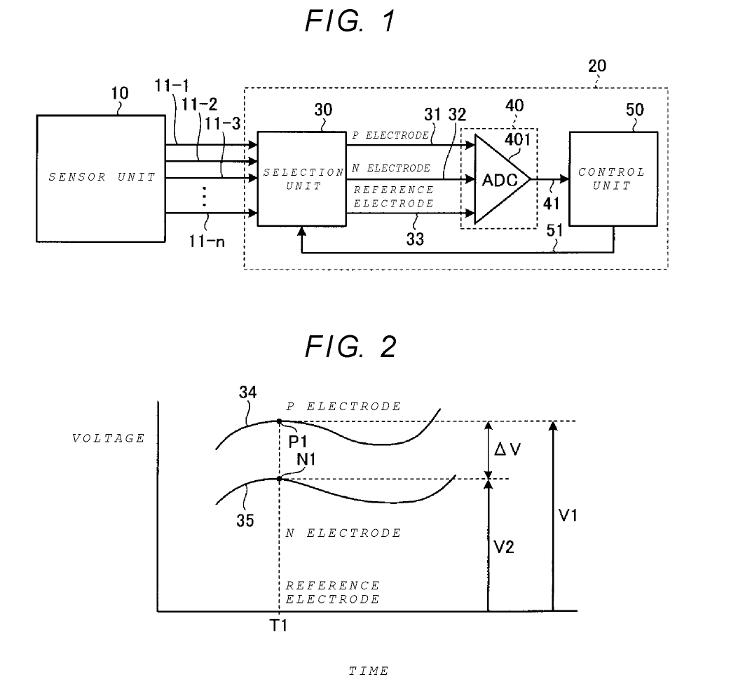

FIG. 1 is a diagram of a myoelectricity measurement device according to a first embodiment. The myoelectricity measurement device includes a sensor unit 10 and a signal processing unit 20. The sensor unit 10 includes a plurality of myoelectricity measurement sensor electrodes. The sensor electrodes come into contact with a skin surface of a user who wears the sensor electrodes to detect a myoelectric signal from the skin surface. The myoelectric signal (hereinafter, referred simply as a potential signal) is generated by contractions of the user's muscles and is detected by the sensor electrodes as a voltage difference between the sensor electrodes. The potential signals are supplied to the signal processing unit 20 via signal lines 11-1 to 11-n (where n is an integer equal to or greater than 3).

The signal processing unit 20 includes a selection unit 30, a measurement unit 40, and a control unit 50. The selection unit 30 selects at least three signal lines from the signal lines 11. The selection unit 30 allocates the selected sensor electrodes as a first input electrode (P electrode), a second input electrode (N electrode), and a reference electrode and connects the electrodes to the measurement unit 40 via signal lines 31 to 33. Hereinafter, the first input electrode (P electrode) and the second input electrode (N electrode) may be collectively referred to as measuring sensor electrodes. The signal lines 31, 32, and 33 are allocated to the P electrode, the N electrode, and the reference electrode, respectively.

The measurement unit 40 measures a voltage difference between the P electrode and the N electrode using the potential signal from the signal line 33 as a reference electrode. The measurement unit 40 includes a differential input type analog-to-digital (AD) converter 401. In some embodiments, the measurement unit 40 may include an operational amplifier and an AD converter for measuring a voltage difference between the P electrode and the N electrode.

A measurement result of the measurement unit 40 is supplied to the control unit 50 to be stored. The control unit 50 supplies a control signal to the selection unit 30 via the signal line 51 based on the measurement result. The selection unit 30 switches between different the combinations of the signal lines 11, that is, the combinations of the measurement sensor electrodes, in response to the control signal.

For example, the control unit 50 selects the combination of the measurement sensor electrodes P and N that produces a largest voltage difference. The control unit 50 includes a central processing unit (CPU), for example.

The myoelectricity measurement device includes a selection unit 30 that selects at least three sensor electrodes from the plurality of sensor electrodes and the selected sensor electrodes are allocated as the reference electrode, the first input electrode (the P electrode), and the second input electrode (the N electrode). The selection unit 30 further supplies signals from the sensor electrodes to the measurement unit 40. In accordance with the measurement result in the measurement unit 40, for example, it is possible to select a combination of the measurement sensor electrodes producing the largest voltage difference between the P electrode and the N electrode. That is, it is possible to select a combination of measurement sensor electrodes for an intended myoelectricity measurement. Thus, when a position of the myoelectricity measurement device is shifted, the combination of the measurement sensor electrodes being used for measurement purposes can be changed in accordance with the potential signals from the sensor electrodes. Therefore, it is possible to provide a highly versatile myoelectricity measurement device.

The measurement unit 40 measures a voltage difference between the P electrode and the N electrode using a potential signal in reference to the reference electrode. By the use of the reference electrode, it is possible to reduce noise in potential measurement. A method of measuring a voltage difference using the reference electrode is an effective method for removing noise and is also used in, for example, a right leg drive used to eliminate noise in an electrocardiogram (ECG) circuit. By reducing noise, it is possible to detect not only a large potential signal by a simple muscle contraction but also a small potential signal by a complicated muscle movement with high precision.

FIG. 2 depicts measurement of myoelectricity and a relationship between voltages of the electrodes. The horizontal axis represents a time and the vertical axis represents a voltage. The reference of the voltage indicates a potential signal from the reference electrode, a potential signal from the P electrode is indicated by a curve 34, and a potential signal from the N electrode is indicated by a curve 35. For example, at time T1, a voltage difference ΔV between a voltage V1 of the P electrode indicated by P1 and a voltage V2 of the N electrode indicated by N1 is measured. The voltage measurement is performed in the measurement unit 40 in FIG. 1.

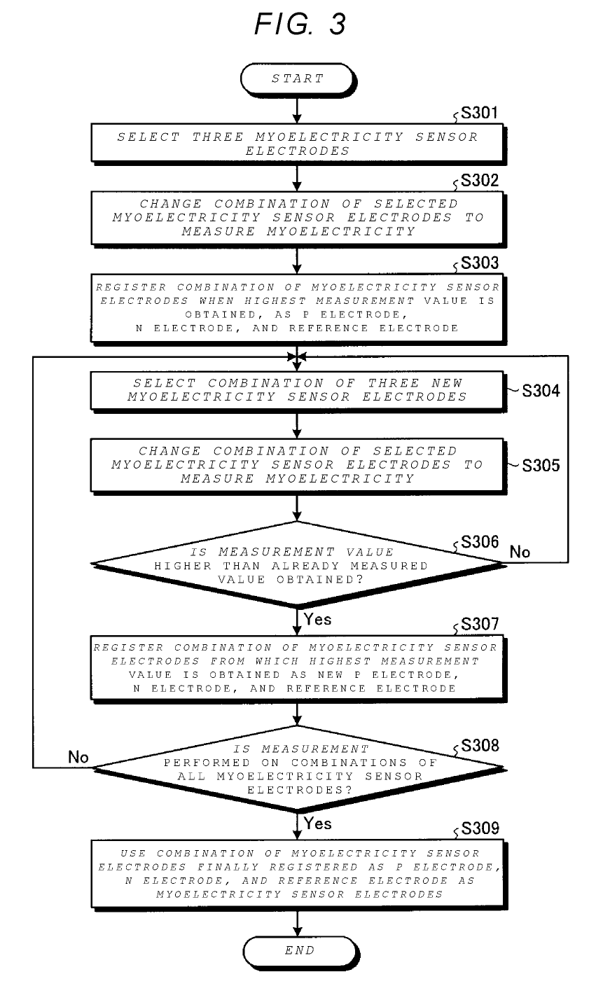

FIG. 3 is a flowchart of a myoelectricity measurement. The myoelectricity measurement is performed in the myoelectricity measurement device in FIG. 1. In FIG. 3, a combination of the measurement sensor electrodes is selected according to a predetermined selection preference. The predetermined selection preference corresponds to a combination of sensor electrodes producing a largest voltage difference.

In S301, the selection unit 30 selects three sensor electrodes that are a first combination of measurement sensor electrodes and a first reference electrode are selected from the plurality of sensor electrodes that are in contact with a skin of a user. The measurement unit 40 measures a voltage difference using the first combination of the measurement sensor electrode.

In S302, the selection unit 30 selects a second combination of measurement sensor electrodes and a second reference electrode. The measurement unit 40 measures a voltage difference using the second combination of the measurement sensor electrode. For example, the sensor electrode selected as the reference electrode in S301 may be selected as the P electrode in S302.

In S303, after steps of S302 are repeated, a combination of sensor electrodes that produces a largest voltage difference among other combinations of sensor electrodes is selected as a combination of measurement sensor electrodes to be used in a subsequent voltage difference measurement. The allocation of the selected measurement sensor electrodes P electrode, the N electrode, and the reference electrode is registered.

In S304, the selection unit 30 selects another set three sensor electrodes. In S305, the newly selected sensor electrodes are allocated as the reference electrode, the P electrode, and the N electrode to be used in a subsequent voltage difference measurement.

In S306, it is determined whether the voltage difference measured by the measurement sensor electrodes selected in S305 is larger than the voltage difference measured by the measurement sensor electrodes previously selected. When the voltage difference measured by the measurement sensor electrodes selected in S305 is not larger (No in S306), the combination of measurement sensor electrodes selected in S305 is used in a subsequent voltage difference measurement, and steps S304 to S306 are repeated. When the voltage difference measured by the measurement sensor electrodes selected in S305 is larger (Yes in S306), in S307, the measurement sensor electrodes selected in S305 are registered as new reference electrode, P electrode, and N electrode for a subsequent voltage difference measurement.

In S308, it is determined whether measurement has been performed by the all combinations of the sensor electrodes. When the measurement by the all combinations of the sensor electrodes has been completed (Yes in S308), the sensor electrodes finally registered as the reference electrode, the P electrode, and the N electrode are used to evaluate muscle movement. When the measurement by the all combinations of the sensor electrodes has not been completed (No in S308), the steps of S304 to S308 are repeated.

As described above, a combination of measurement sensor electrodes can be selected by the selection unit according to measurement of voltage differences by various combinations of sensor electrodes and thus the measurement sensor electrodes that are used for myoelectricity measurement can be appropriately selected.

In the example embodiments described above, the combination of sensor electrodes producing a largest voltage difference was selected for myoelectricity measurement. However, a combination of sensor electrodes for myoelectricity measurement may be selected according to other predetermined selection preferences that are suitable for an intended measurement purpose. The potential signal of each sensor electrode may be stored in a separately provided storage device (not illustrated) and a combination of the sensor electrode may be selected using the value.

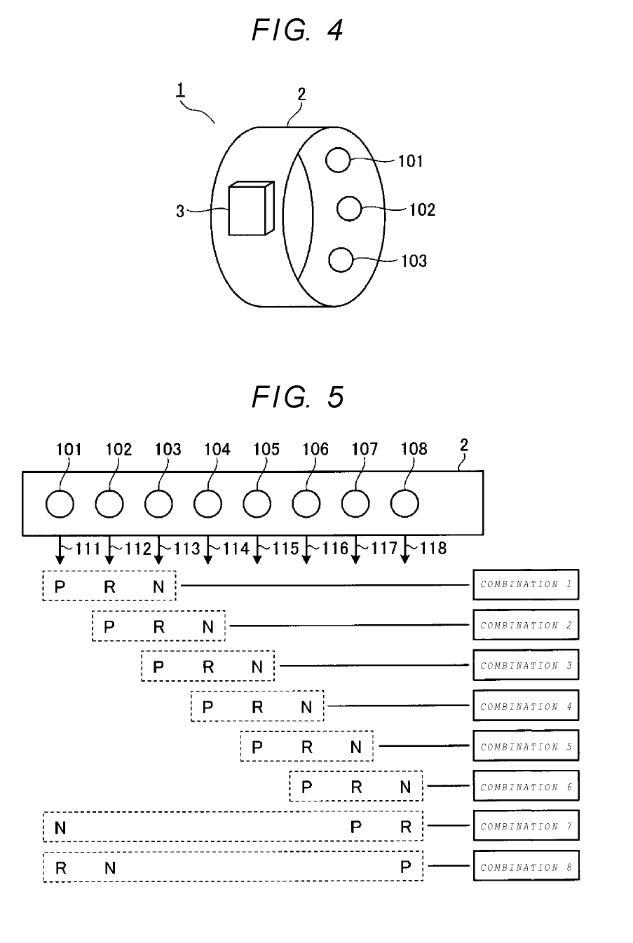

FIG. 4 is a schematic diagram of a myoelectricity measurement device 1. The myoelectricity measurement device 1 includes a holding member 2. The holding member 2 is, for example, an elastic wristband.

The plurality of myoelectricity sensor electrodes 101 to 103 are fixed to the inside of the holding member 2 on the side that is in contact with a skin surface of a user.

A casing 3 is fixed to the outside of the holding member 2. For example, a semiconductor device (not illustrated) including the selection unit 30, the measurement unit 40, and the control unit 50 illustrated in FIG. 1, is accommodated in the casing 3. The sensor electrodes 101 to 103 and the semiconductor device in the casing 3 are connected by wirings (not illustrated) provided in the holding member 2. Potential signals from the sensor electrodes 101 to 103 that are in contact with the skin surface of the user are supplied to the semiconductor device accommodated in the casing 3.

FIG. 5 depicts an enlarged view of the sensor electrodes of the sensor unit 10 illustrated in FIG. 1 and example combinations of measurement sensor electrodes. In FIG. 5, the sensor electrodes are embedded on the holding member 2 and the holding member 2 is illustrated as extended in the horizontal direction for convenience. In the example illustrated in FIG. 5, eight sensor electrodes 101 to 108 are fixed to the holding member 2. The sensor electrodes 101 to 108 have circular shapes and are aligned in a line at an equal interval.

In FIG. 5, eight example selections 1 to 8 of a reference electrode R, and measurement sensor electrodes (P and N) among the sensor electrodes 101 to 108. The potential signals from the sensor electrodes 101 to 108 are supplied to the selection unit 30 via the signal lines 111 to 118. The combinations of the sensor electrodes 101 to 108 are changed by the selection unit 30.

In combination 1, the sensor electrode 101 is allocated as the P electrode, the sensor electrode 102 is allocated as the reference electrode, and the sensor electrode 103 is allocated as the N electrode.

Similarly, in combination 2, the sensor electrode 102 is allocated as the P electrode, the sensor electrode 103 is allocated as the reference electrode, and the sensor electrode 104 is allocated as the N electrode.

In combination 3, the sensor electrode 103 is allocated as the P electrode, the sensor electrode 104 is allocated as the reference electrode, and the sensor electrode 105 is allocated as the N electrode.

In combination 4, the sensor electrode 104 is allocated as the P electrode, the sensor electrode 105 is allocated as the reference electrode, and the sensor electrode 106 is allocated as the N electrode.

In combination 5, the sensor electrode 105 is allocated as the P electrode, the sensor electrode 106 is allocated as the reference electrode, and the sensor electrode 107 is allocated as the N electrode.

In combination 6, the sensor electrode 106 is allocated as the P electrode, the sensor electrode 107 is allocated as the reference electrode, and the sensor electrode 108 is allocated as the N electrode.

In combination 7, the sensor electrode 107 is allocated as the P electrode, the sensor electrode 108 is allocated as the reference electrode, and the sensor electrode 101 is allocated as the N electrode.

In combination 8, the sensor electrode 108 is allocated as the P electrode, the sensor electrode 101 is allocated as the reference electrode, and the sensor electrode 102 is allocated as the N electrode. Each combination is changed by the selection unit 30 in accordance with a control signal from the control unit 50.

For example, a myoelectricity measurement is performed using a set of the reference electrode, the P electrode, and the N electrode that are appropriately selected at a time accordance with voltage differences measured by combinations 1 to 8.

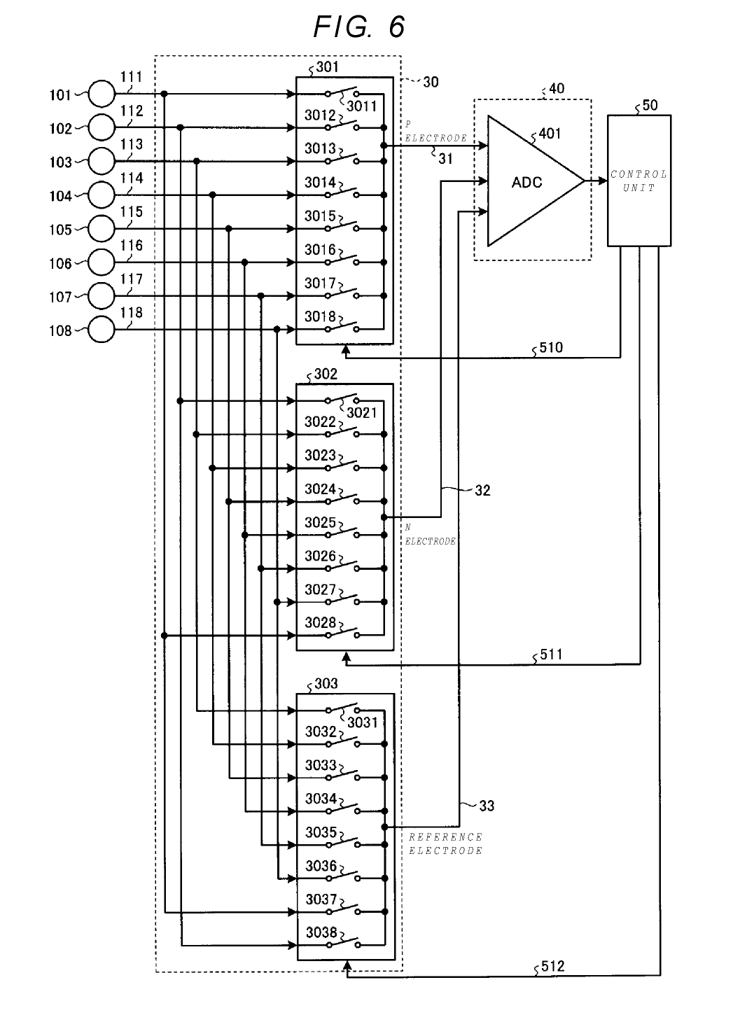

FIG. 6 depicts an example circuit of the selection unit 30. The same reference numerals are used for components that are substantially the same as in the above-described embedment. The sensor electrodes 101 to 108 are connected to the selection unit 30 via the signal lines 111 to 118.

The selection unit 30 includes selection circuits 301, 302, and 303. The selection circuit 301 includes switches 3011 to 3018 connected between the signal lines 111 to 118 and the signal line 31. A control signal supplied from the control unit 50 via a signal line 510 is used to control the switch state of the switches 3011 to 3018. By turning the switches 3011 to 3018 on and off, the particular sensor electrodes 101 to 108 that are allocated as the P electrode are selected. For example, when the switch 3011 is turned on, the sensor electrode 101 is connected to the signal line 31, and thus is allocated as the P electrode.

Similarly, the selection circuit 302 includes switches 3021 to 3028 connected between the signal lines 111 to 118 and the signal line 32. A control signal supplied from the control unit 50 via a signal line 511 is used to control the switch state of the switches 3021 to 3028. For example, when the switch 3021 is turned on, the sensor electrode 102 is connected to the signal line 32, and thus is allocated as the N electrode.

The selection circuit 303 includes switches 3031 to 3038 connected between the signal lines 111 to 118 and the signal line 33. A control signal supplied from the control unit 50 via a signal line 512 is used to control the switches 3031 to 3038. For example, when the switch 3031 is turned on, the sensor electrode 103 is connected to the signal line 33, and thus is allocated as the reference electrode.

By using the control signal to selectively switch the switches (3011 to 3018, 3021 to 3028, and 3031 to 3038) on and off, it is possible to change active combinations of the sensor electrodes 101 to 108.

The selection circuits 301 to 303 can also include or comprise a multiplexer that selects one input from eight inputs and then outputs the selected input.

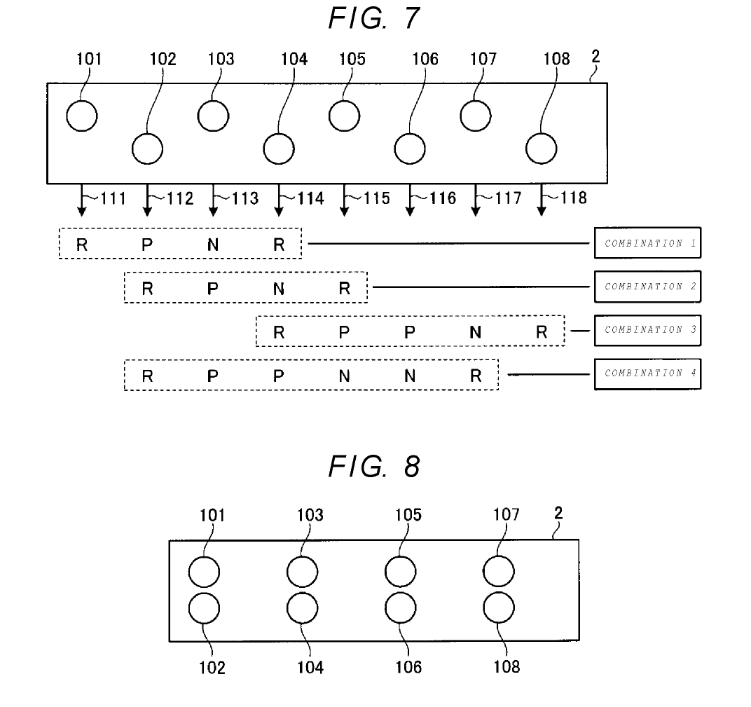

FIG. 7 depicts another example arrangement of the sensor electrodes in a zigzag form and example combinations of measurement sensor electrode. In FIG. 7, the sensor electrodes 101 to 108 are disposed in a zigzag form on the holding member 2. When the sensor electrodes 101 to 108 are disposed in a zigzag form, a position variation among different combinations of the sensor electrodes 101 to 108 can be larger since the positional variation is in the width direction of the holding member in addition to the longitudinal direction of the holding member 2. Accordingly, the most appropriate combination can be effectively selected from the sensor electrodes disposed in such arrangements. In FIG. 7, four example selections 1 to 4 of a reference electrode R, and measurement sensor electrodes (P N) among the sensor electrodes 101 to 108.

For example, in combination 1, the sensor electrodes 101 and 104 are allocated as the reference electrodes, the sensor electrode 102 is allocated as the P electrode, and the sensor electrode 103 is allocated as the N electrode.

That is, in combination 1, the four sensor electrodes 101 to 104 are selected. The sensor electrodes 101 and 104 allocated as the reference electrodes are connected to the signal line 33 for the reference electrode to be selected, for example, by turning the switches 3032 and 3037 illustrated in FIG. 6 on.

Similarly, in combination 2, the sensor electrodes 102 and 105 are allocated as the reference electrodes, the sensor electrode 103 is allocated as the P electrode, and the sensor electrode 104 is allocated as the N electrode.

In combination 3, the sensor electrodes 104 and 108 are allocated as the reference electrodes, the sensor electrodes 105 and 106 are allocated as the P electrodes, and the sensor electrode 107 is allocated as the N electrode.

In combination 4, the sensor electrodes 102 and 107 are allocated as the reference electrodes, the sensor electrodes 103 and 104 are allocated as the P electrodes, and the sensor electrodes 105 and 106 are allocated as the N electrode.

By appropriately combining the number of sensor electrodes which are combination targets, the disposition positions of the sensor electrodes allocated as the reference electrodes, and the like, it is possible to select a combination of the sensor electrodes appropriate for intended myoelectricity measurement. Since the myoelectricity measurement device in FIG. 1 includes the selection unit 30 capable of appropriately selecting the sensor electrodes 101 to 108, it is easy to select the sensor electrodes and change the combination of the sensor electrodes.

FIG. 8 depicts another example arrangement of the sensor electrodes. In FIG. 8, pairs of sensor electrodes (101 and 102, 103 and 104, 105 and 106, and 107 and 108) are disposed in the longitudinal direction of the holding member 2.

The position variations are in the width direction of the holding member 2 in addition to the longitudinal direction of the holding member 2. By providing the pairs of sensor electrodes in the longitudinal direction of the holding member 2, it is possible to provide improved tolerance to position deviation of the holding member 2.

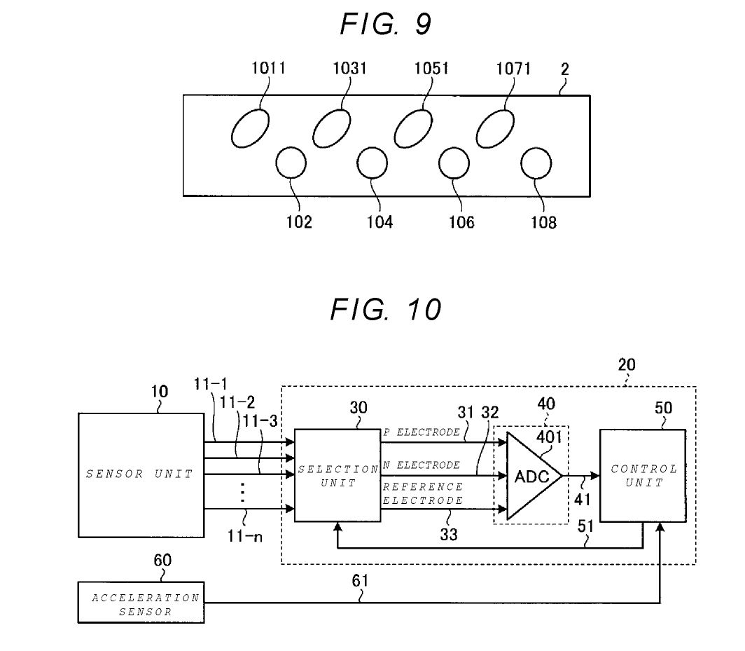

FIG. 9 depicts still another example arrangement of the sensor electrodes. In FIG. 9, the sensor electrodes are disposed in a zigzag form as in the example arrangement of FIG. 7. However, the sensor electrodes (1011, 1031, 1051, and 1071) disposed on the upper side are elliptically shaped and have dimensions greater than the sensor electrodes (102, 104, 106, and 108) disposed on the lower side.

For example, the generated myoelectricity signal will detectably differ for simple motions of the hand, such as making “rock”, “scissors”, and “paper” gestures, and more complicated motions of the hand. When attempting to detect a myoelectricity signal generated in a complicated hand motion, it may be preferred for the sensor electrodes to be small or closely spaced. With sensor electrodes having different shapes or sizes depending on the locations of the sensor electrodes, it is possible to provide a highly versatile myoelectricity measurement device.

In FIG. 9, the sensor electrodes have two kinds of shapes. However, in some embodiments, sensor electrodes may be in more than two kinds of shapes. The myoelectricity measurement device in FIG. 1 includes the selection unit 30 that can appropriately select a combination of measurement sensor electrodes. Therefore, it is possible to increase sensor variation by increasing the kinds of shapes of the sensor electrodes as well as providing sensor electrodes in different positions.

### Second Embodiment

FIG. 10 is a diagram of a myoelectricity measurement device according to a second embodiment. The same reference numerals are used for the components that are substantially the same as those of the first embodiment, and the description of repeated components may be omitted.

In the second embodiment, an acceleration sensor 60 is provided. An output signal of the acceleration sensor 60 is supplied to the control unit 50 via a signal line 61. For example, the acceleration sensor 60 is a triaxial acceleration sensor that detects a direction of acceleration due gravity in the vertical direction.

By calibrating a positional relation of the acceleration sensor 60 with respect to a user prior to a myoelectricity measurement and reflecting a calibration result in a myoelectricity measurement result of the measurement unit 40, it is possible to improve precision in evaluation of muscle movement. A signal of the acceleration sensor 60 may be used as a reference signal for selecting a combination of measurement sensor electrodes to be used for myoelectricity measurement.

While certain embodiments have been described, these embodiments have been presented by way of example only, and are not intended to limit the scope of the inventions. Indeed, the novel embodiments described herein may be embodied in a variety of other forms. Furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Various additional examples of an apparatus configurations and a myoelectricity measurement method are described in the following.

In a myoelectricity measurement device, a measurement unit can include a differential input type AD converter. In a myoelectricity measurement device, a selection unit can include: a first selection circuit that allocates a myoelectricity sensor electrode as a reference electrode among the plurality of myoelectricity sensor electrodes, a second selection circuit that allocates a myoelectricity sensor electrode as a first input electrode among the plurality of myoelectricity sensor electrodes, and a third selection circuit that allocates a myoelectricity sensor electrode as a second input electrode among the plurality of myoelectricity sensor electrodes. A holding member can be formed of an elastic substance in some examples.

A holding member can have a wristband shape that holds the plurality of myoelectricity sensors.

A myoelectricity measurement method of an example embodiment includes: selecting at least three myoelectricity sensor electrodes from the plurality of myoelectricity sensor electrodes; measuring potential signals from the selected myoelectricity sensor electrodes; switching combinations of the myoelectricity sensor electrodes selected from the plurality of myoelectricity sensor electrodes; specifying a desired combination of myoelectricity sensor electrodes among the combinations of the selected myoelectricity sensor electrodes; and measuring myoelectricity by the specified combination of the myoelectricity sensor electrodes.

What is claimed is:

1. A myoelectricity measurement device, comprising:
a plurality of myoelectricity sensor electrodes;
a measurement unit connectable to the plurality of myoelectricity sensor electrodes; and
a control unit configured to:
select a first combination of at least three myoelectricity sensor electrodes from the plurality of myoelectricity sensor electrodes to be connected to the measurement unit;
designate a first electrode in the first combination as a reference electrode, a second electrode in the first combination as a first input electrode, and third electrode in the first combination as a second input electrode;
acquire a first voltage difference between the first and second input electrodes in reference to a voltage of the reference electrode, the voltage difference being measured by the measurement unit; and
select a second combination of at least three myoelectricity sensor electrodes from the plurality of myoelectricity sensor electrodes to be connected to the measurement unit, at least one of the three myoelectricity sensor electrodes in the second combination being different from the at least three myoelectricity sensor electrodes in the first combination.

2. The myoelectricity measurement device according to claim 1, further comprising:
a plurality of signal lines, a first signal line of the plurality of signal lines being connected to the designated first input electrode, a second signal line of the plurality of signal lines being connected to the designated second input electrode, and a third signal line of the plurality of signal lines being connected to the designated third input electrode.

3. The myoelectricity measurement device according to claim 1, further comprising:
a holding member to which the plurality of myoelectricity sensor electrodes is attached.

4. The myoelectricity measurement device according to claim 3, wherein the plurality of myoelectricity sensor electrodes is disposed in a zigzag arrangement on the holding member.

5. The myoelectricity measurement device according to claim 3, wherein the plurality of myoelectricity sensor electrodes is disposed in a line on the holding member.

6. The myoelectricity measurement device according to claim 3, wherein the plurality of myoelectricity sensor electrodes is disposed in two parallel lines on the holding member.

7. The myoelectricity measurement device according to claim 3, wherein the plurality of myoelectricity sensor electrodes includes sensor electrodes that have different shapes.

8. The myoelectricity measurement device according to claim 1, further comprising:
an acceleration sensor;
wherein the control unit selects the at least three electrodes in the second combination based on an output signal from the measurement unit and an output signal from the acceleration sensor.

9. A myoelectricity measurement device, comprising:
a plurality of myoelectricity sensor electrodes;
a plurality of signal lines connected to the plurality of myoelectricity sensor electrodes;
a plurality of switching elements on the plurality of signal lines;
a measurement unit connected to the plurality of signal lines; and
a control unit configured to:
select a first combination of at least three myoelectricity sensor electrodes from the plurality of myoelectricity sensor electrodes by supplying a first control signal to the plurality of switching elements causing the at least three myoelectricity sensor electrodes of the first combination to be connected to the measurement unit;
set a first sensor electrode in the first combination as a first input electrode, a second sensor electrode in the first combination as a second input electrode, and a third sensor electrode in the first combination as a reference electrode;
acquire a first voltage difference between the first and second input electrodes in reference to a voltage of the reference electrode, the voltage difference being measured by the measurement unit; and
select a second combination of at least three myoelectricity sensor electrodes from the plurality of myoelectricity sensor electrodes by supplying a second control signal to the plurality of switching elements causing the at least three myoelectricity sensor electrodes of the second combination to be connected to the measurement unit, at least one of the at least three myoelectricity sensor electrodes in the second combination being different from the at least three myoelectricity sensor electrodes in the first combination.

10. The myoelectricity measurement device according to claim 9, wherein the control unit selects a combination of at least three sensor electrodes for the first combination based on a predetermined selection for a myoelectricity measurement.

11. The myoelectricity measurement device according to claim 9, further comprising:
a plurality of signal lines, a first signal line of the plurality of signal lines being connected to the designated first input electrode, a second signal line of the plurality of signal lines being connected to the designated second input electrode, and a third signal line of the plurality of signal lines being connected to the designated third input electrode.

12. The myoelectricity measurement device according to claim 9, further comprising:
a holding member to which the plurality of myoelectricity sensor electrodes is attached.

13. The myoelectricity measurement device according to claim 12, wherein the plurality of myoelectricity sensor electrodes is disposed in a zigzag arrangement on the holding member.

14. The myoelectricity measurement device according to claim 12, wherein the plurality of myoelectricity sensor electrodes is disposed in a line on the holding member.

15. The myoelectricity measurement device according to claim 12, wherein the plurality of myoelectricity sensor electrodes is disposed in two parallel lines on the holding member.

16. The myoelectricity measurement device according to claim 12, wherein the plurality of myoelectricity sensor electrodes includes sensor electrodes that have different shapes.

17. The myoelectricity measurement device according to claim 9, further comprising:
an acceleration sensor;
wherein the control unit selects the at least three electrodes in the second combination based on an output signal from the measurement unit and an output signal from the acceleration sensor.

18. A myoelectricity measurement device comprising:
a plurality of myoelectricity sensor electrodes;
a plurality of signal lines connected to the plurality of myoelectricity sensor electrodes;
a plurality of switching elements on the plurality of signal lines;
a measurement unit connected to the plurality of signal lines;
a control unit configured to:
select a first combination of at least three myoelectricity sensor electrodes from the plurality of myoelectricity sensor electrodes by supplying a first control signal to the plurality of switching elements causing the at least three myoelectricity sensor electrodes of the first combination to be connected to the measurement unit;
set a first sensor electrode in the first combination as a first input electrode, a second sensor electrode in the first combination as a second input electrode, and a third sensor electrode in the first combination as a reference electrode;
acquire a first voltage difference between the first and second input electrodes in reference to a voltage of the reference electrode, the voltage difference being measured by the measurement unit; and
select a second combination of at least three myoelectricity sensor electrodes from the plurality of myoelectricity sensor electrodes by supplying a second control signal to the plurality of switching elements causing the at least three myoelectricity sensor electrodes of the second combination to be connected to the measurement unit, at least one of the at least three myoelectricity sensor electrodes in the second combination being different from the at least three myoelectricity sensor electrodes in the first combination; and
an elastic wristband to which the plurality of myoelectricity sensor electrodes is attached.

19. The myoelectricity measurement device according to claim 18, wherein the plurality of myoelectricity sensor electrodes includes sensor electrodes that have different shapes.

20. The myoelectricity measurement device according to claim 18, further comprising:
an acceleration sensor;
wherein the control unit selects the at least three electrodes in the second combination based on an output signal from the measurement unit and an output signal from the acceleration sensor.
